(19) United States
(12) Patent Application Publication
(10) Pub. No.: US 2025/0328819 A1
(43) Pub. Date: Oct. 23, 2025

(54) TRAINED MODEL GENERATION METHOD, TRAINED MODEL GENERATION DEVICE, AND RECORDING MEDIUM

(71) Applicant: Panasonic Intellectual Property Management Co., Ltd., Osaka (JP)

(72) Inventors: Maho SHIOTANI, Osaka (JP); Fumi OKUNO, Osaka (JP)

(21) Appl. No.: 19/257,758

(22) Filed: Jul. 2, 2025

Related U.S. Application Data
(63) Continuation of application No. PCT/JP2023/040753, filed on Nov. 13, 2023.
(60) Provisional application No. 63/438,867, filed on Jan. 13, 2023.

(30) Foreign Application Priority Data
Sep. 4, 2023 (JP) 2023-142857

(51) Int. Cl. G06N 20/00; G16H 50/30

(52) U.S. Cl. CPC G06N 20/00; G16H 50/30

(57) ABSTRACT

A trained model generation method is a method for generating a trained model that uses, as an input, a feature based on activity data on a subject and outputs an anomaly score indicating a degree of an anomaly in a physical condition of the subject. A first trained model is a model that has been trained using first activity data on the subject. The first activity data is obtained during a first period. The trained model generation method includes: obtaining the first activity data on the subject and second activity data on the subject, the second activity data being obtained during a second period that is after the first period; and causing a second trained model to be generated using third activity data on the subject when a difference exists between the first activity data and the second activity data, the third activity data being obtained most recently.

### CROSS REFERENCE TO RELATED APPLICATIONS

This is a continuation application of PCT International Application No. PCT/JP2023/040753 filed on Nov. 13, 2023, designating the United States of America, which is based on and claims priority of Japanese Patent Application No. 2023-142857 filed on Sep. 4, 2023 and U.S. Provisional Patent Application No. 63/438,867 filed on Jan. 13, 2023. The entire disclosures of the above-identified applications, including the specifications, drawings and claims are incorporated herein by reference in their entirety.

### FIELD

The present disclosure relates to a trained model generation method, a trained model generation device, and a recording medium.

### BACKGROUND

The 2025 problem in Japan is an aging society problem in which all eight million people belonging to what is called “Dankai no Sedai” (the baby boomer generation) will reach the ages of 75 or older, resulting in a quarter of the nation's population reaching the ages of 75 or older. This problem involves a problem of a labor shortage caused by increasing demands for medical and caregiving services.

Against this backdrop, the number of subjects of nursing and care who are looked after by health care workers and caregivers is increasing. As a result, a small change in a physical condition that may lead to an anomaly in physical condition may be overlooked. If a small change in a physical condition is overlooked, there is a risk that a subject may become more severely ill.

To deal with this, for example, Patent Literature (PTL) 1 discloses a technique of notifying an appropriate recipient of an anomaly in a monitored person when an anomaly in the monitored person is determined. Accordingly, an anomaly of the monitored person can be notified to an appropriate monitoring person in accordance with an anomaly state of the monitored person.

### CITATION LIST

### Patent Literature

PTL 1: WO 2018/116830

### SUMMARY

### Technical Problem

However, in the case where a chronic change in a physical condition has occurred in a subject, the technique according to PTL 1 described above may decrease an anomaly detection performance.

In view of the above, the present disclosure provides a trained model generation method, a trained model generation device, and a recording medium capable of assisting in detecting an anomaly in a subject with high precision in the case where a chronic change in a physical condition has occurred in the subject.

### Solution to Problem

A trained model generation method according to an aspect of the present disclosure is a method for generating a trained model that uses, as an input, a feature based on activity data on a subject and outputs an anomaly score indicating a degree of an anomaly in a physical condition of the subject. In the trained model generation method, a first trained model is a model that has been trained using first activity data on the subject, the first activity data being obtained during a first period in the past. The trained model generation method includes: obtaining the first activity data on the subject and second activity data on the subject, the second activity data being obtained during a second period that is after the first period; determining whether a difference exists between the first activity data and the second activity data; and causing a second trained model to be generated using third activity data on the subject when a difference exists between the first activity data and the second activity data, the third activity data being obtained most recently.

A trained model generation device according to an aspect of the present disclosure is a device that generates a trained model that uses, as an input, a feature based on activity data on a subject and outputs an anomaly score indicating a degree of an anomaly in a physical condition of the subject. In the trained model generation device, a first trained model is a model that has been trained using first activity data on the subject, the first activity data being obtained during a first period in the past. The trained model generation device includes: an obtainer that obtains the first activity data on the subject and second activity data on the subject, the second activity data being obtained during a second period that is after the first period; a determiner that determines whether a difference exists between the first activity data and the second activity data; and a model updater that causes a second trained model to be generated using third activity data on the subject when a difference exists between the first activity data and the second activity data, the third activity data being obtained most recently.

A recording medium according to an aspect of the present disclosure is a non-transitory computer-readable recording medium having recorded thereon a program for causing a computer to execute the trained model generation method described above.

### Advantageous Effects

According to an aspect of the present disclosure, it is possible to implement a trained model generation method and the like capable of assisting in detecting an anomaly in a subject with high precision in the case where a chronic change in a physical condition has occurred in the subject.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating an example of a configuration of a physical condition detection system according to an embodiment.

FIG. 2 is a block diagram illustrating an example of a functional configuration of an information management server according to an embodiment.

FIG. 3 is a flowchart illustrating operation of generating a first trained model by an information management server according to an embodiment.

FIG. 4 is a flowchart illustrating operation of detecting a sign of an anomaly in a physical condition of a subject in an information management server according to an embodiment.

FIG. 5 is a diagram showing an example of graded scores in five levels and conditions for the graded scores according to an embodiment.

FIG. 6 is a flowchart illustrating operation of generating a second trained model by an information management server according to an embodiment.

FIG. 7 is a timing chart schematically illustrating operation of generating a second trained model by an information management server according to an embodiment.

FIG. 8 is a graph illustrating a result of comparison in successful detection rate according to whether to switch the trained models.

FIG. 9 is a first diagram for describing a problem in a conventional method.

FIG. 10 is a second diagram for describing the problem in the conventional method.

### DESCRIPTION OF EMBODIMENTS

### (Circumstances Leading to the Present Disclosure)

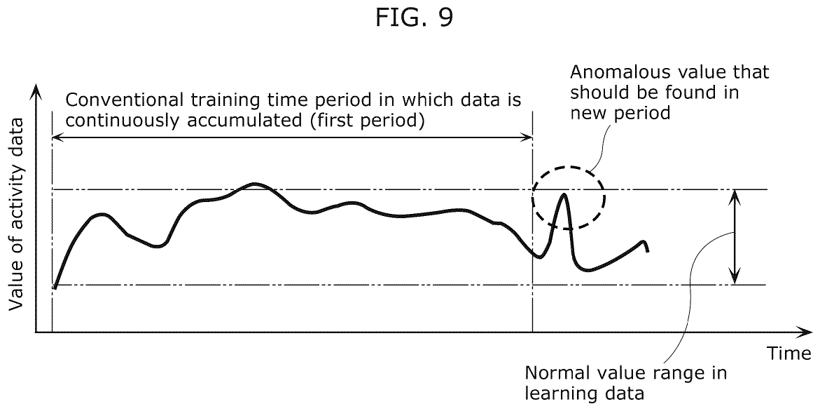

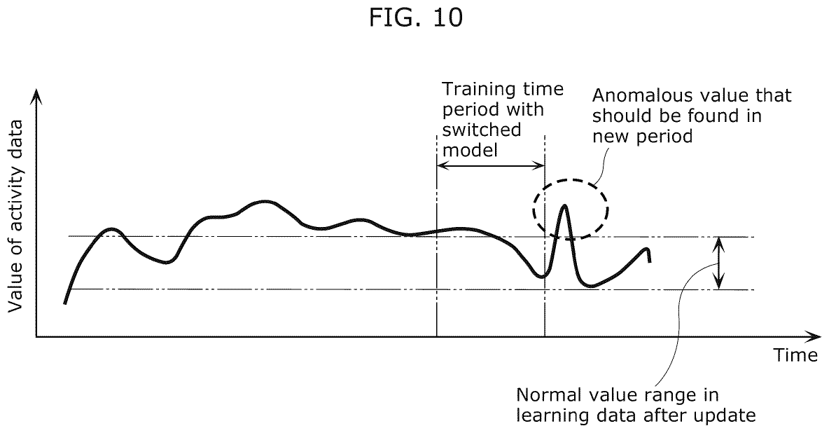

Prior to the description of the present disclosure, the circumstances leading to the present disclosure will be described with reference to FIG. 9 and FIG. 10. FIG. 9 is a first diagram for describing a problem in a conventional method. FIG. 10 is a second diagram for describing the problem in the conventional method.

Generating a machine learning model that detects a small change in the physical condition of a subject leading to an anomaly in the physical condition based on the subject's activity data, care records, and the like has been investigated, which will be described in detail later. The machine learning model may be generated through supervised learning or unsupervised learning. For example, using the supervised learning can generate a trained model that can detect a small change in a physical condition with higher precision than using the unsupervised learning. To use the supervised learning, learning data for performing the supervised learning is collected, and the trained model is generated through the supervised learning using the collected learning data. For example, to generate the learning data, activity data on the subject, such as a respiratory rate or a heart rate is collected.

Here, in the case of an elderly person in need of care, or the like, a chronic change may occur in a physical condition (e.g., activity data) (a change in the physical condition) due to an influence of disease or aging. In a conventional model building method, all collected and accumulated activity data is used as the learning data. Thus, for example, the learning data may include activity data before and after the occurrence of a chronic change in a physical condition or may include only activity data before the occurrence of a chronic change in a physical condition. A trained model generated with such activity data may affect the detection performance (specifically, decrease the detection performance).

Note that the chronic change in a physical condition is a gradual, continuous change in a body condition and can occur due to factors such as age, lifestyle, genetic factors, or obesity. For example, the chronic change in a physical condition may be a gradual change in a body condition that spans several weeks or several months. The chronic change in a physical condition does not include a sudden change in a physical condition (e.g., a sudden headache, a sudden high fever, etc.).

As illustrated in FIG. 9, a trained model is generated using learning data accumulated in a training time period (a first period) during which data is continuously accumulated, and the trained model is used to detect a small change in a physical condition leading to an anomaly in the physical condition of a subject. Assume that the range between dashed-double dotted lines is set at this time as a normal value range in the learning data. Note that the first period is assumed to be a period including a time point at which a chronic change in a physical condition (such a change in the physical condition that the value of activity data continuously decreases) occurs in the subject and time points before and after the time point. That is, the learning data includes activity data items before and after the occurrence of the chronic change in the physical condition in the subject. It is considered that the activity data items before and after the occurrence of the chronic change in the physical condition differ in the normal value range.

Assume that the chronic change in the physical condition of the subject also continues after the first period, and that, for example, a small change in the physical condition occurs within a time slot enclosed with a broken-line frame (“Anomalous value that should be found in new period” in FIG. 9). Although the small change is prominent in the activity data, using the trained model trained with the activity data in the first period may lead to such a determination that the time point of the prominence is normal because of the normal value range set based on the activity data in the first period.

Hence, as illustrated in FIG. 10, in a case where a chronic change in a physical condition has occurred in a subject, it is desired to provide a training time period for accumulating data to be used to switch (rebuild) the trained model (“Training time period with switched model” in FIG. 10, a second period) and to rebuild the trained model using the data in the second period. That is, in the case where a chronic change in a physical condition has occurred in a subject, it is desired to generate a new trained model using the data collected in the second period. The second period is a period including the chronic change in the physical condition. By rebuilding the trained model using activity data obtained in the second period, it is possible to reset the normal value range (“Normal value range in learning data after update” in FIG. 10) in the case where the chronic change in the physical condition has occurred. Note that the length of the second period is not limited to a particular length. The second period may be shorter than the first period, may be the same as the first period, or may be longer than the first period.

In the case where the anomalous value (the prominence in the broken-line frame illustrated in FIG. 10) as illustrated in FIG. 9 occurs after the normal value range is updated, using the updated normal value range makes it possible to improve the reliability of detecting the occurrence of a small change in a physical condition at the time point of the prominence. That is, it is considered that using the updated trained model makes it possible to detect a small change in a physical condition with higher precision in the case where a subject has a chronic change in the physical condition.

PTL 1 does not disclose a technique of detecting a small change in a physical condition in a subject with higher precision in the case where a chronic change in the physical condition has occurred in the subject.

Hence, the inventors of the present application conducted diligent studies about assisting in detecting a small change in a physical condition (a sign of an anomaly in the physical condition) in a subject with higher precision in the case where a chronic change in the physical condition has occurred in the subject and originated a trained model generation method and the like described below.

A trained model generation method according to a first aspect of the present disclosure is a method for generating a trained model that uses, as an input, a feature based on activity data on a subject and outputs an anomaly score indicating a degree of an anomaly in a physical condition of the subject. In the trained model generation method, a first trained model is a model that has been trained using first activity data on the subject, the first activity data being obtained during a first period in the past. The trained model generation method includes: obtaining the first activity data on the subject and second activity data on the subject, the second activity data being obtained during a second period that is after the first period; determining whether a difference exists between the first activity data and the second activity data; and causing a second trained model to be generated using third activity data on the subject when a difference exists between the first activity data and the second activity data, the third activity data being obtained most recently.

Accordingly, in the case where the difference exists between the first activity data and the second activity data, that is, in the case where it is considered that the chronic change in the physical condition has occurred in the subject, the trained model that outputs the anomaly score is updated. The updated trained model (a second trained model) is trained with most recent third activity data. Thus, the updated trained model can be a model that is adapted to the most recent state of the subject (i.e., the state in which the chronic change in the physical condition has occurred). Therefore, in the trained model generation method, using the updated model makes it possible to assist in detecting an anomaly in the subject in the case where the chronic change in the physical condition has occurred in the subject with higher precision. Note that the updating includes generating a new trained model.

For example, a trained model generation method according to a second aspect is the trained model generation method according to the first aspect, in which the third activity data may include the second activity data.

Accordingly, the second trained model is trained with the current state of the subject (latest activity data). Thus, it is possible to generate a model that can detect whether a chronic change in a physical condition has occurred in the current state of the subject with high precision. Thus, it is possible to assist in detecting an anomaly in the subject with further higher precision.

For example, a trained model generation method according to a third aspect is the trained model generation method according to the second aspect, in which the third activity data may further include part of the first activity data.

Accordingly, in the case where learning data for generating the second trained model is insufficient, it is possible to complement the insufficiency with learning data in the first period. Thus, it is possible to prevent the detection performance of the second trained model from decreasing due to insufficient learning data.

For example, a trained model generation method according to a fourth aspect is the trained model generation method according to any one of the first through third aspects, in which the difference may include a difference indicating that the subject has a chronic change in the physical condition.

Accordingly, it is possible to update the model at a timing when the chronic change in the physical condition occurs.

For example, a trained model generation method according to a fifth aspect is the trained model generation method according to any one of the first through fourth aspects, in which the determining of whether the difference exists between the first activity data and the second activity data may be performed using a statistic of the first activity data and a statistic of the second activity data.

Accordingly, using the statistic makes it possible to easily determine whether the subject has the chronic change in the physical condition.

For example, a trained model generation method according to a sixth aspect is the trained model generation method according to any one of the first through fifth aspects, in which the difference may be determined to exist when Expression 1 below is satisfied:

$$( \vert {sd}_{A} - s {d}_{B} \vert / {sd}_{A} ) \times 100 \geq threshold \qquad ( Expression 1 )$$

where sd_{B} denotes a standard deviation of the first activity data and sd_{A} denotes a standard deviation of the third activity data.

Accordingly, substituting the standard deviation into Expression 1 makes it possible to easily determine whether the subject has the chronic change in the physical condition. In addition, using the standard deviation makes it possible to easily detect a chronic change in the physical condition, that is, a chronic change in the activity data.

For example, a trained model generation method according to a seventh aspect is the trained model generation method according to any one of the first through sixth aspects, in which the determining of whether the difference exists between the first activity data and the second activity data may be performed every predetermined period.

Accordingly, the determination as to whether to update the model is performed every predetermined period. Thus, for example, even in the case where a chronic change in a physical condition occurs in the future, it is possible to assist in detecting an anomaly in the subject with higher precision.

For example, a trained model generation method according to an eighth aspect is the trained model generation method according to any one of the first through seventh aspects, and may further include: generating the second trained model when a predetermined incident involving the subject occurs.

Accordingly, the second trained model is generated based on the incident that may lead to the occurrence of the change in the physical condition in the subject. Thus, it is possible to assist in detecting an anomaly in the subject with higher precision even in the case where no chronic change in the physical condition has occurred.

For example, a trained model generation method according to a ninth aspect is the trained model generation method according to any one of the first through eighth aspects, and may further include: switching the trained model used for outputting the anomaly score to the subject, from the first trained model to the second trained model, when the second trained model is generated.

Accordingly, in the case where the chronic change in the physical condition has occurred in the subject, the trained model is switched to the second trained model, which is suitable for the change in the physical condition. Thus, it is possible to assist in detecting an anomaly in the subject with higher precision.

For example, a trained model generation method according to a tenth aspect is the trained model generation method according to any one of the first through ninth aspects, in which the first trained model may be continuously used when no difference exists between the first activity data and the second activity data.

Accordingly, by continuously using the first trained model, it is possible to assist in detecting an anomaly in the subject in the case where no chronic change in a physical condition has occurred in the subject. In addition, the model is not updated in the case where no chronic change in a physical condition has occurred. Thus, it is possible to reduce the load on a device that executes the trained model generation method.

A trained model generation device according to an eleventh aspect of the present disclosure is a device that generates a trained model that uses, as an input, a feature based on activity data on a subject and outputs an anomaly score indicating a degree of an anomaly in a physical condition of the subject. In the trained model generation device, a first trained model is a model that has been trained using first activity data on the subject, the first activity data being obtained during a first period in the past. The trained model generation device includes: an obtainer that obtains the first activity data on the subject and second activity data on the subject, the second activity data being obtained during a second period that is after the first period; a determiner that determines whether a difference exists between the first activity data and the second activity data; and a model updater that causes a second trained model to be generated using third activity data on the subject when a difference exists between the first activity data and the second activity data, the third activity data being obtained most recently. In addition, a recording medium according to a twelfth aspect of the present disclosure is a non-transitory computer-readable recording medium having recorded thereon a program for causing a computer to execute the trained model generation method according to any one of the first through tenth aspects.

Accordingly, it is possible to produce the same advantageous effects as those produced by the trained model generation method described above.

Note that these general and specific aspects may be implemented using a system, a method, an integrated circuit, a computer program, or a non-transitory computer-readable recording medium such as a compact disc read-only memory (CD-ROM), or any combination of systems, methods, integrated circuits, computer programs, or recording media. The program(s) may be stored in a recording medium in advance or may be supplied to a recording medium via a wide area communication network including the Internet, for example.

Hereinafter, exemplary embodiments will be specifically described with reference to the drawings.

Note that each of the exemplary embodiments described below shows a general or specific example. The numerical values, constituent elements, the arrangement and connection of the constituent elements, steps, the processing order of the steps etc. shown in the following exemplary embodiments are mere examples, and therefore do not intend to limit the present disclosure. Among the constituent elements in the following exemplary embodiments, those not recited in any one of the independent claims will be described as optional constituent elements.

The drawings are represented schematically and are not necessarily precise illustrations. Thus, the scales, for example, are not necessarily consistent from drawing to drawing. In the drawings, constituent elements that are substantially the same are given the same reference signs, and redundant descriptions will be omitted or simplified.

In the present specification, numerical values and numerical value ranges do not express the strict meanings only, but also include substantially equivalent ranges, e.g., differences of several percent (or about 10%).

In the present specification, ordinal numerals such as “first” and “second” do not mean the number or order of constituent elements unless otherwise stated in particular. The ordinal numerals are used to avoid confusion of and distinguish between constituent elements of the same type.

### Embodiment

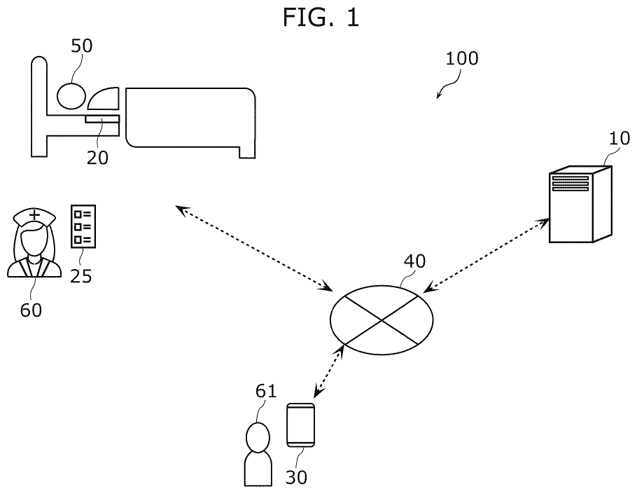

Hereinafter, a trained model generation method and the like according to the present embodiment will be described with reference to FIG. 1 through FIG. 8.

### [1. Configuration of Physical Condition Detection System]

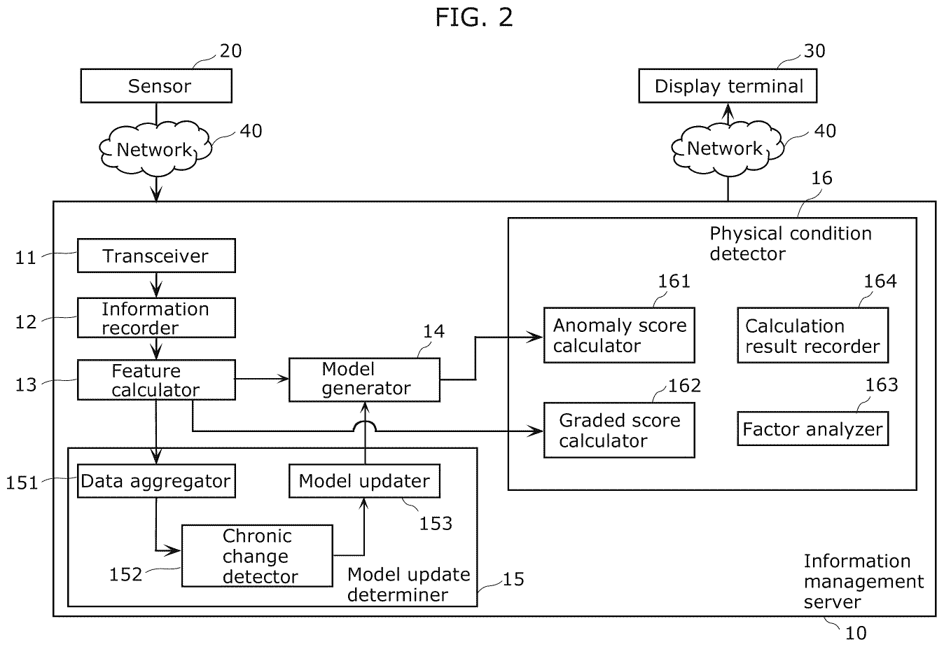

First, a configuration of a physical condition detection system including an information management server that executes the trained model generation method will be described with reference to FIG. 1 and FIG. 2. FIG. 1 is a diagram illustrating an example of a configuration of physical condition detection system 100 according to the present embodiment.

Physical condition detection system 100 according to the present embodiment is a system configured such that information management server 10 detects a small change in the physical condition of subject 50 of nursing or care that may lead to an anomaly in the physical condition of subject 50 (i.e., a sign of an anomaly in the physical condition).

As illustrated in FIG. 1, physical condition detection system 100 includes information management server 10, sensor 20, and display terminal 30. These are communicably connected together via communication network 40. Communication network 40 may be a wired network, may be a wireless network, or may be both a wired network and a wireless network. FIG. 1 also illustrates subject 50 of nursing or care, user 60 who is an on-site staff member such as a health care worker who provides nursing or care to subject 50, user 61 who can check display terminal 30 and is an on-site staff member such as a monitoring person who monitors subject 50, and recorded data 25 that includes recorded details of nursing or care of subject 50 by user 60. On recorded data 25, for example, food intakes, body temperatures, and the like that are input by user 60 being an on-site staff member are recorded. The food intakes are amounts of food taken by subject 50 in the morning, afternoon, evening, and the like

Note that although FIG. 1 illustrates an example of the case where physical condition detection system 100 includes one sensor 20, this is not limiting, and it suffices if physical condition detection system 100 includes as many sensors 20 as subjects 50 of nursing or care.

Sensor 20 obtains activity data on subject 50 during a predetermined time period by sensing. The activity data includes at least one of a heart rate, a respiratory rate, in/out of bed, a body temperature, or a food intake. The activity data may include at least two of the heart rate, the respiratory rate, the in/out of bed, the body temperature, or the food intake. For example, sensor 20 may obtain data on the heart rate, the respiratory rate, a body motion, and the like (hereinafter, also referred to as sensor data) every second while subject 50 is in bed. Sensor 20 may further sense whether subject 50 is in or out of bed according to whether sensor 20 can sense the heart rate, the respiratory rate, the body motion, and the like.

Note that an interval at which the sensor data including the heart rate, the respiratory rate, the body motion, and the like is obtained is not limited to one second. The interval may be two seconds. The interval may be an interval in any unit so long as it enables sensing of changes in the sensor data of subject 50. Sensor 20 may further sense a life rhythm such as a sleep state according to whether sensor 20 can sense the heart rate, the respiratory rate, the body motion, and the like.

Note that the following will mainly describe the case where the activity data includes the respiratory rate and the heart rate.

For example, sensor 20 may be a sensor device having a pressure sensor or the like and may be placed in a bed to sense subject 50 every second. In this case, for example, sensor 20 may output, every second, the value “1” indicating being out of bed as sensor data indicating that subject 50 is out of bed. For example, sensor 20 may output sensor data such as the respiratory rate of subject 50 every second.

Sensor 20 may be, for example, an image capturing device such as a camera and is provided such that sensor 20 can capture subject 50 who is in bed or subject 50 who is eating. The camera may be a thermal camera that detects the body temperature of subject 50 or may be a normal camera (e.g., a charge coupled device (CCD) camera). The food intakes may be obtained by performing image analysis on images.

Information management server 10 is implemented using, for example, a computer including a processor (a microprocessor), a memory, a communication interface, and the like. Information management server 10 may be configured to operate with a part of the configuration of information management server 10 included in a cloud server. Information management server 10 generates a trained model for detecting a small change in a physical condition of subject 50 that may lead to an anomaly in the physical condition of subject 50 (i.e., a sign of an anomaly in the physical condition) and uses the generated trained model to detect the small change in the physical condition of subject 50 that may lead to the anomaly in the physical condition of subject 50.

FIG. 2 is a block diagram illustrating an example of a functional configuration of information management server 10 according to the present embodiment.

As illustrated in FIG. 2, information management server 10 includes transceiver 11, information recorder 12, feature calculator 13, model generator 14, model update determiner 15, and physical condition detector 16. At least model update determiner 15 constitutes a trained model generation device. Note that the trained model generation device may be implemented as a stand-alone device.

Transceiver 11 includes, for example, a communication interface and transmits and receives various types of information to and from sensor 20 or display terminal 30 via communication network 40. Transceiver 11, for example, obtains activity data including the respiratory rate and the heart rate of subject 50 during a predetermined time period. Here, the activity data may include, for example, at least the respiratory rate and the heart rate among the body temperature, food intake, the respiratory rate, the heart rate, and an out-of-bed rate of subject 50 during the predetermined time period. Further, transceiver 11 outputs the graded score calculated by physical condition detector 16 to a terminal possessed by user 61 such as a monitoring person who monitors subject 50.

In the present embodiment, transceiver 11 obtains the sensor data such as the heart rate, the respiratory rate, and the body motion per second while subject 50 is in bed, from sensor 20 via communication network 40 at predetermined intervals, for example, every minute. Transceiver 11 also obtains, for example, recorded data 25 that includes recorded details of nursing or care for subject 50 by user 60 being an on-site staff member as illustrated in FIG. 1. In this manner, transceiver 11 obtains activity data that includes the sensor data and recorded data 25 and is obtained on-site every day, via communication network 40.

Transceiver 11 also transmits a graded score calculated by physical condition detector 16 to display terminal 30 via communication network 40. Note that transceiver 11 may transmit information for a display that is presented on a user interface of display terminal 30 to cause user 61 to handle an anomaly in the physical condition of subject 50, such as a graded score display, a vital fluctuation graph display, or a risk group display described later. Transceiver 11 may also obtain at least one of the body temperature or the food intake of subject 50 that are included in the care record.

Information recorder 12 records information transmitted and received by transceiver 11. Information recorder 12 is a recording medium capable of recording information and includes, for example, a rewritable, nonvolatile memory such as a hard disk drive and a solid state drive. Note that information recorder 12 may record a plurality of features calculated by feature calculator 13.

Feature calculator 13 includes, for example, a computer including a memory and a processor (microprocessor). With the processor executing a control program stored in the memory, feature calculator 13 implements a function of calculating the plurality of features. Feature calculator 13 calculates the plurality of features based on the activity data including respiratory rates and heart rates of subject 50 that are obtained by transceiver 11. For example, feature calculator 13 obtains sensor data for a time period including a target date and time of detecting the physical condition from activity data obtained by transceiver 11 or recorded on information recorder 12 and calculates hourly features for each type of sensor data such as the respiratory rate.

Here, feature calculator 13 may calculate, for example, at least a mean value and a maximum value of respiratory rates of subject 50 and a mean value and a maximum value of heart rates of subject 50, as a plurality of hourly features. In the present embodiment, feature calculator 13 calculates, from at least the respiratory rates and the heart rates, the mean values and the maximum values of the respiratory rates and the heart rates as the plurality of features, from among mean values, maximum values, standard deviations, skewnesses, kurtoses, and impulse factors of the respiratory rates, difference data on the respiratory rates, the heart rates, and difference data on the heart rates. Here, each of the impulse factors is obtained by subtracting the corresponding mean value from the corresponding maximum value. In this manner, feature calculator 13 performs statistical processing and the like on the activity data to calculate the plurality of features.

More specifically, feature calculator 13 calculates, for example, respiratory-rate-related features and heart-rate-related features of subject 50 on an hourly basis. For example, feature calculator 13 obtains sensor data indicating respiratory rates of subject 50 within a time period including a target date and time of detecting the physical condition from activity data recorded on information recorder 12 or sensor data obtained from sensor 20 and calculates hourly statistical features for the time period.

In more detail, feature calculator 13 obtains, for example, respiratory rate data on respiratory rates not being zero during a given hour from the activity data and calculates, as the statistical features, a mean value, a maximum value, a minimum value, a standard deviation, a skewness, a kurtosis, an impulse factor, and the like during the hour from the obtained respiratory rate data. Here, the impulse factor can be calculated from a difference between the maximum value and the mean value (maximum value-mean value) of the respiratory rate data for the hour. Feature calculator 13 also calculates, as the statistical features, a mean value, a maximum value, a minimum value, a standard deviation, a skewness, a kurtosis, an impulse factor, and the like during the hour from the difference data on the obtained respiratory rate data. The difference data on the obtained respiratory rate data is data indicating, for example, a difference between a respiratory rate at time point t and a respiratory rate at a time point t+1, which is one second after time point t, that is, differences between items of the respiratory rate data on a per-second basis. Note that it suffices if feature calculator 13 calculates, as the statistical features, at least the mean value and the maximum value during the hour from the obtained respiratory rate data.

For example, feature calculator 13 also obtains heart rate data indicating heart rates of subject 50 within a time period including a target date and time of detecting the physical condition from the activity data recorded on information recorder 12 or the sensor data obtained from sensor 20 and calculates hourly statistical features for the time period.

Here, feature calculator 13 obtains, for example, heart rate data on heart rates not being zero during a given hour from the activity data and calculates, as the statistical features, a mean value, a maximum value, a minimum value, a standard deviation, a skewness, a kurtosis, an impulse factor, and the like during the hour from the obtained heart rate data. Feature calculator 13 also calculates, as the statistical features, a mean value, a maximum value, a minimum value, a standard deviation, a skewness, a kurtosis, an impulse factor, and the like during the hour from difference data on the obtained heart rate data. The difference data on the obtained heart rate data is data indicating, for example, a difference between a heart rate at time point t and a heart rate at a time point t+1, which is one second after time point t, that is, differences between items of the heart rate data on a per-second basis, as with the difference data on the respiratory rate data. Note that it suffices if feature calculator 13 calculates, as the statistical features, at least the mean value and the maximum value during the hour from the obtained heart rate data.

Note that feature calculator 13 may calculate one of the food intake, the out-of-bed rate, or the body temperature of subject 50 as one of the plurality of features.

For example, feature calculator 13 may calculate the food intake of subject 50 from care records included in the activity data, as one of the plurality of features. In this case, it suffices if feature calculator 13 calculates a total amount of food intakes in one day in the past from the care records and next calculates a total sum of food intakes within a time period including a target date and time of detecting the physical condition. Here, in the case where target dates and times are set in morning, afternoon, and night time periods, it suffices if feature calculator 13 calculates, for example, a total sum of food intakes during a period from the morning of a previous day of the target date of detecting the physical condition to the morning of the target date, a period from the afternoon of the previous day to the afternoon of the target date, and a period from the night of the previous day to the night of the target date.

For example, feature calculator 13 may also calculate an out-of-bed rate from the activity data obtained by transceiver 11 and recorded on information recorder 12, as one of the plurality of features. In this case, it suffices if feature calculator 13 obtains in-or-out-of-bed data indicating whether subject 50 is in or out of bed within a time period including a target date and time of detecting the physical condition from the activity data recorded on information recorder 12 or the sensor data obtained from sensor 20 and calculates an hourly out-of-bed rate for the time period. In more detail, feature calculator 13 can calculate an out-of-bed rate during a given hour by, for example, counting up the number of values “1” indicating being out of bed during the hour and dividing the number by a total number during the hour (i.e., a total of the number of values “1” indicating being out of bed and the number of values “0” indicating being in bed during the hour).

Alternatively, feature calculator 13 may calculate, for example, the body temperature of subject 50 from care records included in the activity data, as one of the plurality of features. In this case, it suffices if feature calculator 13 calculates a body temperature (e.g., a mean body temperature) for one day in the past from the care records and next calculates a body temperature (e.g., a mean body temperature) for a time period including a target date and time of detecting the physical condition. Here, in the case where target dates and times are set in morning, afternoon, and night time periods, it suffices if feature calculator 13 calculates, for example, body temperatures during a period from the morning of a previous day of the target date of detecting the physical condition to the morning of the target date, a period from the afternoon of the previous day to the afternoon of the target date, and a period from the night of the previous day to the night of the target date.

Model generator 14 generates the model using the plurality of features. Model generator 14 may generate the model (a supervised model) through supervised learning using learning data that includes information based on the plurality of features and training data. Model generator 14 may generate the model (an unsupervised model) that has learned normality or anomaly through unsupervised learning using learning data that includes an activity data group including the plurality of features. When the model generated in this manner receives a plurality of features of subject 50, the model outputs an anomaly score indicating a degree of an anomaly in the physical condition of subject 50. Note that the following will mainly describe an example of generating the model through the supervised learning.

In the present embodiment, model generator 14 includes a computer including, for example, a memory and a processor (a microprocessor), and implements various functions by means of the processor executing a control program stored in the memory. Model generator 14 obtains activity data in a training time period from activity data recorded on information recorder 12 or sensor data obtained from sensor 20. Note that model generator 14 may obtain recorded data 25 in the training time period and add recorded data 25 in the training time period to the activity data in the training time period.

Model generator 14 also causes feature calculator 13 to calculate hourly features based on the activity data in the training time period. Using the hourly features in the training time period, model generator 14 trains the model, thus generating the model.

As the supervised learning model, for example, an anomaly detection with generative adversarial network (AnoGAN), a variational autoencoder (VAE), a deep support vector data description (Deep SVDD), or the like is used. However, this is not limiting.

As the unsupervised learning model, a model that separates an outlier based on a decision tree (e.g., an Isolation Forest model), a model based on K-means clustering (k-means), or the like is used. However, this is not limiting.

Model generator 14, for example, may generate a model specific to subject 50. That is, there are as many models as subjects 50.

Model update determiner 15 determines, based on the obtained activity data, whether a chronic change in a physical condition has occurred in subject 50. In the case where the chronic change in the physical condition has occurred in subject 50, model update determiner 15 causes model generator 14 to generate a new model. Model update determiner 15, for example, may use at least activity data obtained after the generation of the model by model generator 14 to determine whether the chronic change in the physical condition has occurred.

Model update determiner 15 is implemented using a computer including, for example, a processor (a microprocessor), memory, a communication interface, and the like, and implements various functions by means of the processor executing a control program stored in the memory.

Model update determiner 15 includes data aggregator 151, chronic change detector 152, and model updater 153.

Data aggregator 151 aggregates first activity data on subject 50 obtained during a first period and second activity data on subject 50 obtained during a second period, which is after the first period. Data aggregator 151 aggregates the first activity data, which is used to generate a model currently used by physical condition detector 16 (an example of a first trained model), and aggregates the second activity data, which is obtained during a period that is after the generation of the model and includes a most recent predetermined period. Data aggregator 151 collects, from feature calculator 13, a first feature based on the first activity data and a second feature based on the second activity data. However, data aggregator 151 may collect the first activity data and the second activity data themselves. Data aggregator 151 is an example of an obtainer. The aggregating means accumulating data, for example, collecting data and storing the data in a storage.

Note that the most recent predetermined period refers to a period from a time point a predetermined period before a current time point to the current time point. An example of the most recent predetermined period is the past one month.

Chronic change detector 152 detects whether a chronic change in a physical condition has occurred in subject 50 based on the first activity data and the second activity data aggregated by data aggregator 151. For example, chronic change detector 152 may determine whether a difference of at least a threshold exists between the first activity data and the second activity data, and when determining that the difference of at least the threshold exists, chronic change detector 152 may detect that the chronic change in the physical condition has occurred in subject 50. As the threshold, a value indicating that subject 50 has a chronic change in the physical condition is set in advance.

The determining of whether the difference exists between the first activity data and the second activity data may be performed, for example, using a statistic of the first activity data and a statistic of the second activity data. For example, the determining of whether the difference exists between the first activity data and the second activity data may be performed using the difference between mean values of the statistics (e.g., Expression 1 shown below) or may be performed using a significance test.

When sd_{B} denotes a standard deviation of the first activity data and sd_{A} denotes a standard deviation of most recent second activity data, chronic change detector 152 determines that the difference exists between the activity data items when Expression 1 shown below is satisfied.

$$( \vert {sd}_{A} - s {d}_{B} \vert / {sd}_{A} ) \times 100 \geq threshold \qquad ( Expression 1 )$$

Standard deviations sd_{A} and sd_{B} are examples of the statistic.

In the case where the respiratory rate is used as the activity data, a larger value is set to the threshold than in the case where the heart rate is used as the activity data. For example, the threshold may be set to be 10%, 15%, or 20%. In the case where the heart rate is used as the activity data, a smaller value is set to the threshold than in the case where the respiratory rate is used as the activity data. For example, the threshold may be set to be 3%, 5%, or 7%. The threshold may be set in advance and recorded by model update determiner 15.

Using Expression 1, chronic change detector 152 may perform the determination on each of a plurality of activity data items (a plurality of features), and may detect that the chronic change in the physical condition has occurred in subject 50 when Expression 1 is not satisfied in at least one of the activity data items (at least one of the features).

Note that chronic change detector 152 may determine whether a predetermined incident involving subject 50 has occurred and determine to generate the second trained model when determining that a predetermined incident has occurred. The predetermined incident is an incident relating to the physical condition of subject 50. Examples of the predetermined incident include entering and leaving a hospital. Chronic change detector 152 is an example of the determiner.

When chronic change detector 152 detects the chronic change in the physical condition in subject 50, that is, when the difference exists between the first activity data and the second activity data, model updater 153 causes model generator 14 to generate a second trained model, which has been trained with a third feature based on third activity data on subject 50 most recently obtained. The third activity data may include at least the second activity data. The third activity data may further include part of the first activity data. The part of the first activity data is data in a period closer to now (a newer period) in the first period. The part may be used in the case where, for example, the second period, during which the second activity data is obtained, is shorter than a predetermined period, that is, in the case where the number of second activity data items is small. For example, model updater 153 determines whether the number of second activity data items is at least a predetermined number. When the number of second activity data items is less than the predetermined number, model updater 153 may add only the part of the first activity data to the third activity data. Note that when the number of second activity data items is less than the predetermined number, all the first activity data items are not added to the third activity data.

When model generator 14 obtains model generating instructions from model updater 153, model generator 14 changes learning data for generating the model from previous one and generates a new model. Specifically, model generator 14 generates the new model (the second trained model) using the third activity data.

Physical condition detector 16 is implemented using a computer including, for example, a processor (a microprocessor), memory, a communication interface, and the like, and implements various functions by means of the processor executing a control program stored in the memory. Physical condition detector 16 detects an anomaly in the physical condition of subject 50, using the model generated by model generator 14 and the plurality of features calculated by feature calculator 13.

Physical condition detector 16 includes anomaly score calculator 161, graded score calculator 162, factor analyzer 163, and calculation result recorder 164.

Anomaly score calculator 161 inputs the plurality of features calculated by feature calculator 13 into the model generated by model generator 14 to obtain an anomaly score indicating a degree of an anomaly in a physical condition per predetermined period.

In the present embodiment, anomaly score calculator 161 inputs a plurality of hourly features on a target date of detection of the physical condition of subject 50 that are calculated by feature calculator 13 into the supervised model generated by model generator 14. Anomaly score calculator 161 records hourly anomaly scores on the target date of detection of the physical condition of subject 50 that are calculated, on calculation result recorder 164.

Based on the anomaly score calculated by anomaly score calculator 161, graded score calculator 162 calculates a graded score that indicates a physical condition anomaly level of subject 50 in a graded manner.

In the present embodiment, graded score calculator 162 calculates a daily anomaly score mean value from hourly anomaly scores of a target date of detecting the physical condition that are recorded on calculation result recorder 164 or calculated by anomaly score calculator 161. Likewise, graded score calculator 162 calculates daily anomaly score mean values on a previous day of the target date of detecting the physical condition and on the day before the previous day, from hourly anomaly scores on the previous day of the target date and on the day before the previous day that are recorded on calculation result recorder 164. Graded score calculator 162 totalizes the daily anomaly score means on the target date, the previous day, and the day before the previous day, thus calculating a three-day total score. Note that the three-day total score is an example of a total score used in a calculation method for calculating a graded score with high accuracy, and this is not limiting. It suffices if a period for calculating the total score ranges from one day to five days.

Graded score calculator 162 calculates threshold values for graded scores (may also be referred to as graded threshold values) from a three-day total score group for about 90 days in the past from the target date recorded on calculation result recorder 164. More specifically, graded score calculator 162 calculates the graded threshold values by calculating a mean and a standard deviation of the three-day total score group for about 90 days in the past.

Graded score calculator 162 outputs a value of the calculated graded score to calculation result recorder 164. For example, in the case where the graded scores are in five levels, graded score calculator 162 may further output the calculated graded score to display terminal 30 via communication network 40 when the value of the calculated graded score is from one to three.

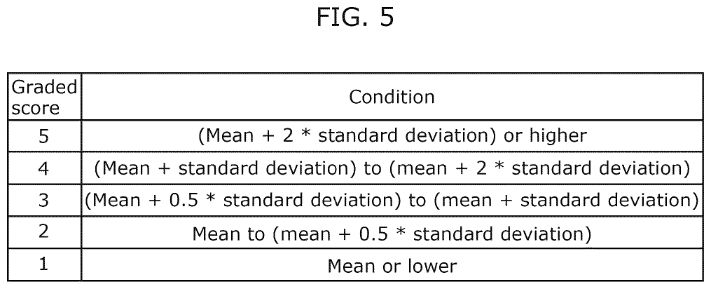

Processing by graded score calculator 162 will be described later with reference to FIG. 5.

Factor analyzer 163 performs factor analysis when the graded score is greater than or equal to a predetermined value to analyze, for each of elements included in the activity data, whether the element is a factor for the graded score being greater than or equal to the predetermined value. Here, the elements include, for example, food intake, the respiratory rate, the heart rate, the body temperature, or an out-of-bed rate of subject 50 during the predetermined time period. In addition, the predetermined value is a value at which handling a sign of an anomaly in a physical condition is needed. For example, in the case where the graded scores are in five levels, the predetermined value may be determined to be four or five, in the case where the graded scores are in three levels, the predetermined value may be determined to be three, and in the case where the graded scores are in two levels, the predetermined value may be determined to be two.

In the present embodiment, in the case where the graded score calculated by graded score calculator 162 is four or five, factor analyzer 163 performs a factor analysis on elements including heart rates, respiratory rates, out-of-bed rates, food intakes, and body temperatures, which are included in activity data used to calculate features. In the case where the activity data used to calculate features includes only the heart rates and the respiratory rates, it suffices if the factor analysis is performed on elements including the heart rates and the respiratory rates.

For example, factor analyzer 163 converts a plurality of features of each element in an entire time period used to calculate a graded score into data of a plurality of daily features of the element to calculate mean values and standard deviations in the entire time period used to calculate the graded score. In the present embodiment, factor analyzer 163 converts a plurality of features of each element in three days into data of a plurality of daily features of the element to calculate mean values and standard deviations of the element in the three days.

Factor analyzer 163 then makes an analysis showing that the element does not form a factor when Expression 2 shown below is established, and makes an analysis showing that the element forms a factor when Expression 2 shown below is not established.

$$( Mean value - 2 * standard deviation ) \leq ( feature of the element at target date and time ) \leq ( mean value + 2 * standard deviation ) \qquad ( Expression 2 )$$

Note that Expression 2 uses a nature of standard deviation that 95.45% of all data items are distributed within the range that is twice as much as mean value±standard deviation.

Factor analyzer 163 outputs the graded score and the element analyzed to be a factor by the factor analysis to calculation result recorder 164. Factor analyzer 163 may also output the graded score and the element analyzed to be a factor by the factor analysis to display terminal 30 via communication network 40.

Calculation result recorder 164 is a recording medium capable of recording a calculation result and includes, for example, a rewritable, nonvolatile memory such as a hard disk drive and a solid state drive. In the present embodiment, calculation result recorder 164 records, as the calculation result, the anomaly score calculated by anomaly score calculator 161, the graded score calculated by graded score calculator 162, and the like. Note that calculation result recorder 164 may record a factor analyzed by factor analyzer 163, as the calculation result.

Display terminal 30 is implemented using a computer including, for example, a processor (a microprocessor), memory, a communication interface, a user interface, and the like. Display terminal 30 is a terminal possessed by user 61 such as the monitoring person who monitors subject 50, and is, for example, a mobile terminal device such as a tablet or a smartphone. Display terminal 30 may be a mobile computer or a stationary computer (a stationary terminal device) connected to a display device.

In the present embodiment, display terminal 30 can be checked by user 61 such as the monitoring person who monitors subject 50. Display terminal 30 is connected to communication network 40, and when, for example, a graded score is obtained from information management server 10, causes the user interface to present a display for user 61 to handle an anomaly in the physical condition of subject 50. The user interface can cause a display device to present a display according to, for example, an input from user 61.

### [2. Operation of Information Management Server]

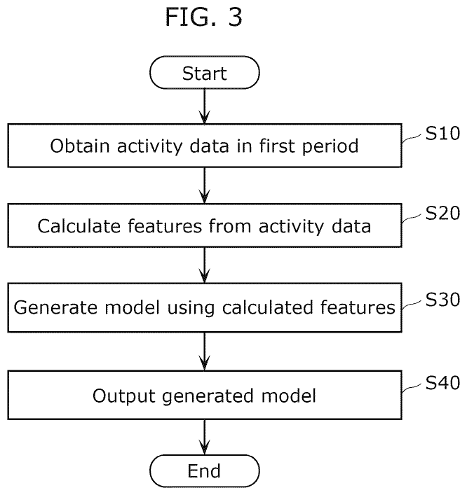

Subsequently, with reference to FIG. 3 to FIG. 8, the operation of information management server 10 configured in the above-described manner will be described. First, with reference to FIG. 3, the operation of generating the model (here, the supervised learning model) for detecting a sign of an anomaly in the physical condition of subject 50 will be described.

FIG. 3 is a flowchart illustrating the operation of generating the first trained model (the trained model generation method) by information management server 10 according to the present embodiment.

As illustrated in FIG. 3, transceiver 11 obtains activity data in the first period from sensor 20 through communication (S10). Transceiver 11, for example, obtains activity data on a per-minute basis. Transceiver 11 obtains sensor data including a heart rate, a respiratory rate, a body motion, or the like, on a per-minute basis. Note that transceiver 11 may collectively obtain the activity data in the first period or may obtain the activity data every time sensor 20 performs sensing.

Next, feature calculator 13 calculates features from the obtained activity data (S20). Feature calculator 13 calculates a statistic of hourly activity data. Feature calculator 13 may, for example, perform statistical processing and the like on hourly heart rates and hourly respiratory rates to calculate one or more statistics. Feature calculator 13 may, for example, perform statistical processing and the like on results of hourly in/out of bed to calculate one or more statistics including an out-of-bed rate. The calculated statistic is an example of a feature.

Feature calculator 13 then generates a plurality of feature sets, with statistics of activity data for 24 hours taken as one set. Feature calculator 13 may, for example, take statistics of heart rates and respiratory rates for 24 hours, and in/out of bed statuses for 24 hours as one set to be used as learning data for a data augmented model.

Next, model generator 14 generates the model using the calculated features (S30). For example, in the case of the supervised learning, model generator 14 trains the model through machine learning using the features as input data and anomaly scores as training data (labeled data).

Next, model generator 14 outputs the generated model to anomaly score calculator 161 (S40). Model generator 14 may record the generated model on a recorder (not illustrated) of physical condition detector 16.

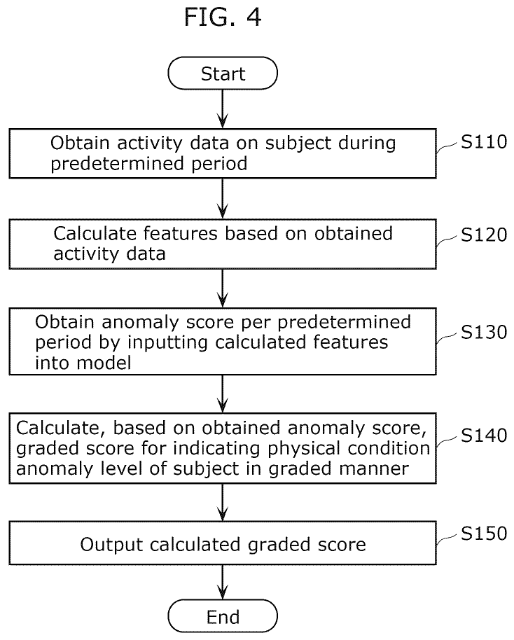

Subsequently, with reference to FIG. 4 and FIG. 5, operation of detecting a sign of an anomaly in the physical condition of subject 50 using the model generated as above will be described.

FIG. 4 is a flowchart illustrating the operation of detecting a sign of an anomaly in the physical condition of subject 50 in information management server 10 according to the present embodiment.

As illustrated in FIG. 4, transceiver 11 obtains activity data on subject 50 (e.g., activity data including respiratory rates and heart rates of subject 50) during a predetermined time period (S110).

Next, feature calculator 13 calculates features based on the activity data obtained in step S110 (S120). Feature calculator 13 calculates, for example, hourly features (e.g., a plurality of hourly features). For example, feature calculator 13 may calculate a plurality of hourly features on a target date of detecting the physical condition of subject 50 based on activity data including at least respiratory rates and heart rates of subject 50 obtained by transceiver 11.

Next, anomaly score calculator 161 obtains an anomaly score per predetermined time period by inputting the features calculated in step S120 into the model that is pretrained (S130). Anomaly score calculator 161 calculates hourly anomaly scores from, for example, the plurality of hourly features. Anomaly score calculator 161 obtains anomaly scores each indicating a degree of an anomaly in the physical condition per hour in a predetermined time period including the target date, by inputting the features calculated by feature calculator 13 into the model generated by model generator 14.

Next, graded score calculator 162 calculates, based on the anomaly score obtained in step S130, a graded score that indicates a physical condition anomaly level of subject 50 in a graded manner (S140). For example, graded score calculator 162 calculates a daily mean value of anomaly scores from the hourly anomaly scores. In the present embodiment, graded score calculator 162 calculates a daily anomaly score mean value from the hourly anomaly scores in the predetermined time period including the target date of detecting the physical condition of subject 50.

Graded score calculator 162 also calculates the graded threshold values by calculating a mean and a standard deviation of past anomaly scores before the target date (e.g., anomaly scores for roughly past 90 days).

FIG. 5 is a diagram showing an example of graded scores in five levels and conditions for the graded scores according to the present embodiment.

As shown in FIG. 5, for example, when calculating the graded scores in five levels, graded score calculator 162 can calculate the threshold values from a mean and a standard deviation. For example, according to the conditions shown in FIG. 5, a threshold value for a graded score of one is the mean, and threshold values for a graded score of two are between the mean and a value that is subtraction of a value being half the standard deviation from the mean.

Graded score calculator 162 then applies the calculated graded threshold values to the mean value of the anomaly scores on the target date, thus calculating the graded score. More specifically, graded score calculator 162 calculates a value of the graded score by subjecting the mean value of the anomaly scores on the target date to determination using the threshold values calculated under the conditions shown in FIG. 5.

Graded score calculator 162 may further check whether the graded score calculated in step S140 indicates a value of four or five, that is, whether a value indicating an anomaly in the physical condition is calculated. In the case where the graded score is the value of four or five, factor analyzer 163 may perform a factor analysis on elements including heart rates, respiratory rates, out-of-bed rates, body temperatures, and food intakes, which are included in the activity data used to calculate the features.

Next, graded score calculator 162 outputs the graded score calculated in step S140 (S150). In the case where an element has been analyzed to be a factor by the factor analysis by factor analyzer 163, graded score calculator 162 may output in step S150 the graded score and the element analyzed to be a factor by the factor analysis.

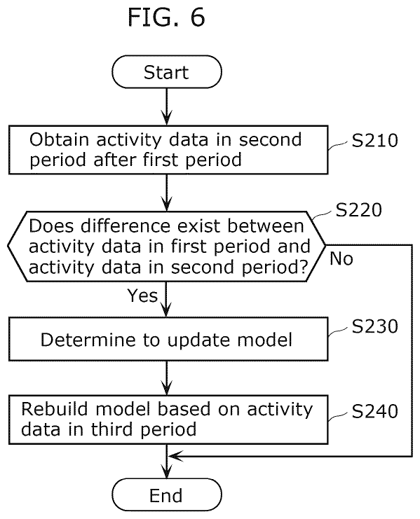

Subsequently, with reference to FIG. 6 to FIG. 8, the operation of updating the model generated in the above manner will be described.

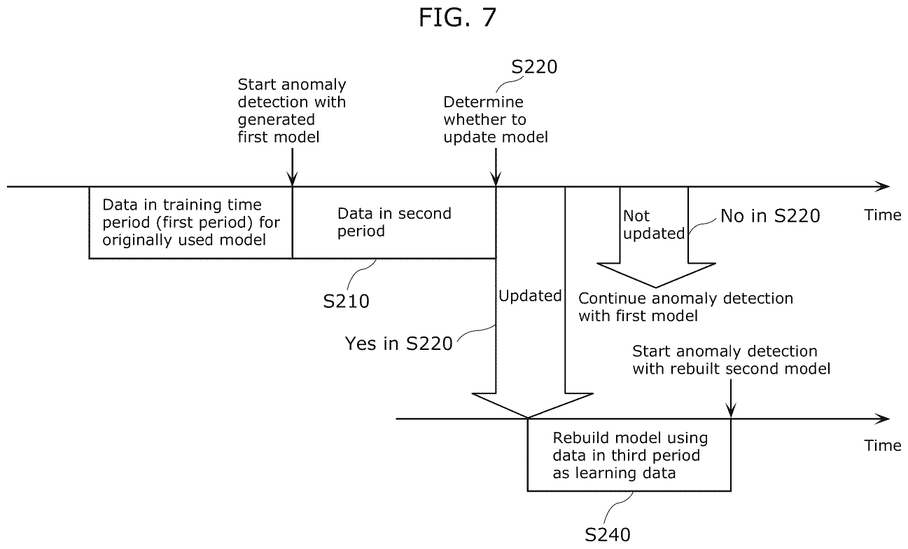

FIG. 6 is a flowchart illustrating the operation of generating the second trained model (the trained model generation method) by information management server 10 according to the present embodiment. FIG. 7 is a timing chart schematically illustrating the operation of generating the second trained model by information management server 10 according to the present embodiment.

The operation illustrated in FIG. 6 is executed after the execution of step S40 illustrated in FIG. 3 or during the execution of the operation of detecting a sign of an anomaly in a physical condition illustrated in FIG. 4. The operation illustrated in FIG. 6 may be executed every predetermined period or may be executed when a predetermined incident involving subject 50 occurs. Note that the predetermined period may be, for example, a period necessary to collect activity data items enough to determine whether a chronic change in a physical condition has occurred in subject 50. The predetermined period is set in advance and recorded on a recorder (not illustrated) of information management server 10.

The “originally used model” illustrated in FIG. 7 means a currently used model (a first model, a first trained model). With consideration given to the case where the model is to be updated, the model is referred to as the “originally used model”.

As illustrated in FIG. 6 and FIG. 7, data aggregator 151 of model update determiner 15 obtains activity data in the second period after the first period from information recorder 12 or feature calculator 13 (S210). The second period may include a period after anomaly detection with the generated first model is started. Data aggregator 151 may also obtain the activity data in the first period. Note that although the first period and the second period are, for example, periods not overlapping in time with each other, the first period and the second period may overlap with each other.

Next, chronic change detector 152 determines whether a difference exists between the activity data in the first period and the activity data in the second period (S220). Chronic change detector 152 performs the determination in step S220 using, for example, Expression 1 shown above. The determination in step S220 is executed every predetermined period in step S210.

Next, when chronic change detector 152 determines that the difference exists (Yes in S220), model updater 153 determines to update the model (S230). Because a chronic change in a physical condition may have occurred in subject 50, model updater 153 determines to generate a new model suitable for the state where the chronic change in the physical condition has occurred.

Model updater 153 next causes model generator 14 to rebuild the model based on activity data in a third period (S240). Model updater 153 gives model generator 14 instructions to regenerate the model using the activity data in the third period, that is, most recent activity data. The third period, for example, is a period for obtaining the activity data used to rebuild the model. The third period may be the same as the second period, may be part of the second period, or may be a period including the second period and part of the first period.

Obtaining the instructions from model updater 153, model generator 14 regenerates a model that supports the chronic change in the physical condition (an example of the second trained model, a second model illustrated in FIG. 7) using the activity data obtained most recently. The regenerated model is output to physical condition detector 16. Then, physical condition detector 16 switches the trained model used for outputting an anomaly score to subject 50, from the first trained model to the second trained model. Accordingly, the effect of improving the precision of detecting a sign of an anomaly in the physical condition of subject 50 in the case where a chronic change in the physical condition has occurred in subject 50 can be expected. That is, with information management server 10, it is possible to detect a sign of an anomaly in the physical condition of subject 50 with higher precision in the case where a chronic change in the physical condition has occurred in subject 50.

Note that when chronic change detector 152 determines that no difference exists (No in S220), physical condition detector 16 finishes the process of updating the model. When no difference exists between the first activity data and the second activity data, that is, when it is considered that chronic change in a physical condition has not occurred in subject 50, the first trained model (the first model) is continuously used.

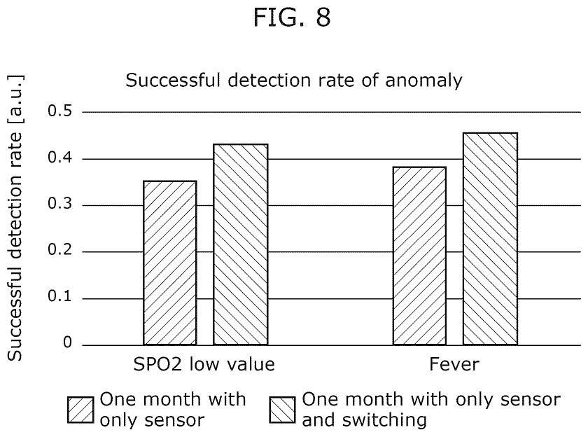

Here, with reference to FIG. 8, a successful detection rate of anomaly in subject 50 according to whether to update the model will be described. FIG. 8 is a graph illustrating a result of comparison in successful detection rate according to whether to switch the trained models. In FIG. 8, successful detection rates are compared between not updating the model (“One month with only sensor” in FIG. 8) and updating the model (“One month with only sensor and switching” in FIG. 8) for saturation pulse O₂ (SPO2: percutaneous oxygen saturation) low value and fever, as the anomaly in a physical condition. Not updating the model indicates a conventional example in which a chronic change in a physical condition has occurred in subject 50 but the model is not updated. Not updating the model indicates the case where the model before the update in the updating the model is continuously used although a chronic change in a physical condition has occurred in subject 50. The updating the model indicates the method according to the present disclosure, in which the model is updated because a chronic change in a physical condition has occurred in subject 50. Note that the vertical axis in FIG. 8 indicates the successful detection rate (0 to 1).

As illustrated in FIG. 8, updating the model improves the successful detection rate for both SPO2 low value and fever.

### Other Embodiments

Although the trained model generation method and so on according to one or more aspects have been described above based on an embodiment, the present disclosure is not limited to the embodiment. The present disclosure may also include forms achieved by making various modifications to the above embodiment that can be conceived by those skilled in the art, as well as forms achieved by combining constituent elements in different embodiments, without materially departing from the spirit of the present disclosure.

For example, in the above embodiment and so on, each of the constituent elements may be configured in the form of an exclusive hardware product, or may be implemented by executing a software program suitable for the constituent element. Each of the constituent elements may be implemented by a program executor, such as a central processing unit (CPU) or a processor, reading and executing the software program recorded on a recording medium such as a hard disk or semiconductor memory.

The processing order of executing the steps shown in the flowcharts is a mere illustration for specifically describing the present disclosure, and thus may be an order other than the shown order. Also, one or more of the steps may be executed simultaneously (in parallel) with another step, or may be steps that are not executed.

The divisions of the functional blocks shown in the block diagram are mere examples, and thus a plurality of functional blocks may be implemented as a single functional block, or a single functional block may be divided into a plurality of functional blocks, or one or more functions may be moved to another functional block. Also, the functions of a plurality of functional blocks having similar functions may be processed by a single hardware or software product in a parallelized or time-divided manner.

The information management server according to the above embodiment and so on may be implemented as a single device or may be implemented by a plurality of devices. When the information management server is implemented by a plurality of devices, the constituent elements included in the information management server may be assigned to the plurality of devices in any manner. When the information management server is implemented by a plurality of devices, the method of communication between the plurality of devices is not particularly limited; the communication may be wireless communication or may be wired communication. Wireless communication and wired communication may be combined for communication between the devices.

Each of the constituent elements described in the above embodiment and so on may be implemented in the form of a software product, or may be typically implemented as a large-scale integrated (LSI) circuit, which is an integrated circuit (IC). These may take the form of individual chips, or may be partially or entirely packaged into a single chip. Although the term “LSI” is used here, other names, such as IC, system LSI, super LSI, and ultra LSI may be used, depending on the level of integration. Furthermore, the manner in which the circuit integration is achieved is not limited to LSI, and it is also possible to use a dedicated circuit (a general-purpose circuit that executes a dedicated program) or a general-purpose processor. A field programmable gate array (FPGA) that allows for programming after the manufacture of an LSI circuit, or a reconfigurable processor that allows for reconfiguration of the connection and the setting of circuit cells inside an LSI circuit may be employed. Furthermore, when advancement in semiconductor technology or derivatives of other technologies brings forth a circuit integration technology which replaces LSI, it will be appreciated that such a circuit integration technology may be used to integrate the constituent elements.

A system LSI circuit is a super-multifunctional LSI circuit manufactured with a plurality of processing units integrated on a single chip, and is specifically a computer system including a microprocessor, read-only memory (ROM), and random-access memory (RAM), for example. A computer program is stored in the ROM. The system LSI circuit achieves its function as a result of the microprocessor operating according to the computer program.

An aspect of the present disclosure may be a computer program that causes a computer to execute each characteristic step included in the trained model generation method illustrated in any one of FIG. 3, FIG. 4, FIG. 6, or FIG. 7.

For example, the program may be a program to be executed by a computer. An aspect of the present disclosure may be a non-transitory computer-readable recording medium having such a program recorded thereon. For example, such a program may be recorded on a recording medium and distributed. For example, by installing the distributed program in a device that includes another processor and causing the processor to execute the program, it is possible to cause the device to perform each of the processes described above.

### INDUSTRIAL APPLICABILITY

The present disclosure is applicable to a trained model generation method for generating a trained model that can assist in detecting a small change in the physical condition of a subject that may lead to an anomaly in the physical condition of the subject.

1. A trained model generation method for generating a trained model that uses, as an input, a feature based on activity data on a subject and outputs an anomaly score indicating a degree of an anomaly in a physical condition of the subject,
wherein a first trained model is a model that has been trained using first activity data on the subject, the first activity data being obtained during a first period in past,
the trained model generation method comprising:
obtaining the first activity data on the subject and second activity data on the subject, the second activity data being obtained during a second period that is after the first period;
determining whether a difference exists between the first activity data and the second activity data; and
causing a second trained model to be generated using third activity data on the subject when a difference exists between the first activity data and the second activity data, the third activity data being obtained most recently.

2. The trained model generation method according to claim 1, wherein
the third activity data includes the second activity data.

3. The trained model generation method according to claim 2, wherein
the third activity data further includes part of the first activity data.

4. The trained model generation method according to claim 1, wherein
the difference includes a difference indicating that the subject has a chronic change in the physical condition.

5. The trained model generation method according to claim 1, wherein
the determining of whether the difference exists between the first activity data and the second activity data is performed using a statistic of the first activity data and a statistic of the second activity data.

6. The trained model generation method according to claim 1, wherein
the difference is determined to exist when Expression 1 below is satisfied:
(|sd_{A}−sd_{B}|/sd_{A})×100≥threshold (Expression 1)
where sd_{B} denotes a standard deviation of the first activity data and sd_{A} denotes a standard deviation of the third activity data.

7. The trained model generation method according to claim 1, wherein
the determining of whether the difference exists between the first activity data and the second activity data is performed every predetermined period.

8. The trained model generation method according to claim 1, further comprising:
generating the second trained model when a predetermined incident involving the subject occurs.

9. The trained model generation method according to claim 1, further comprising:
switching the trained model used for outputting the anomaly score to the subject, from the first trained model to the second trained model, when the second trained model is generated.

10. The trained model generation method according to claim 1, wherein
the first trained model is continuously used when no difference exists between the first activity data and the second activity data.

11. A trained model generation device that generates a trained model that uses, as an input, a feature based on activity data on a subject and outputs an anomaly score indicating a degree of an anomaly in a physical condition of the subject,
wherein a first trained model is a model that has been trained using first activity data on the subject, the first activity data being obtained during a first period in past,
the trained model generation device comprising:
an obtainer that obtains the first activity data on the subject and second activity data on the subject, the second activity data being obtained during a second period that is after the first period;
a determiner that determines whether a difference exists between the first activity data and the second activity data; and
a model updater that causes a second trained model to be generated using third activity data on the subject when a difference exists between the first activity data and the second activity data, the third activity data being obtained most recently.

12. A non-transitory computer-readable recording medium having recorded thereon a program for causing a computer to execute the trained model generation method according to claim 1.
